(19) United States
(12) Patent Application Publication
(10) Pub. No.: US 2022/0357184 A1
(43) Pub. Date: Nov. 10, 2022

(54) ATTACHMENT FOR MONITORING INHALER USAGE

(71) Applicant: INOFAB SAGLIK TEKNOLOJILERI ANONIM SIRKETI, Ankara (TR)

(72) Inventors: Kerem YASAR, Kocaeli (TR); Merthan OZTURK, Ankara (TR); Selim YONET, Ankara (TR); Ahmet OGUZ, Bursa (TR); Kadir Tayyip TASLICA, Ankara (TR); Ceren YAVUZ, Ankara (TR)

(73) Assignee: INOFAB SAGLIK TEKNOLOJILERI ANONIM SIRKETI, Ankara (TR)

(21) Appl. No.: 17/623,638

(22) PCT Filed: Dec. 29, 2020
(86) PCT No.: PCT/TR2020/051441

§ 371 Date: Dec. 29, 2021

(30) Foreign Application Priority Data
Sep. 25, 2020 (TR) 2020/15247

(51) Int. Cl. G01D 5/241; A61M 15/00

(52) U.S. Cl. CPC G01D 5/2417; A61M 15/009; A61M 2205/3317; A61M 2230/40; A61M 2205/13

(57) ABSTRACT

An attachment for monitoring an inhaler usage to be used for monitoring a usage frequency of patients using inhalers is provided. The attachment allows monitoring the inhaler usage of the patients and provides a low energy consumption. With an effective power management system developed with the attachment, the attachment is used for a longer period of time without a need for a battery replacement.

### CROSS REFERENCE TO THE RELATED APPLICATIONS

This application is the national stage entry of International Application No. PCT/TR2020/051441, filed on Dec. 29, 2020, which is based upon and claims priority to Turkish Patent Application No. 2020/15247 filed on Sep. 25, 2020, the entire contents of which are incorporated herein by reference.

### TECHNICAL FIELD

This invention is related to an attachment for monitoring inhaler usage to be used for monitoring the usage frequency of patients using inhalers.

### BACKGROUND

In order to deliver the drugs to the lungs, an inhaler is used, which mixes a drug into the air by allowing the air to be passed through a limited volume during the breathing (inhalation) of the user. The user needs to inhale at a certain rate in order to prevent the drug from reaching the lungs in insufficient amounts and to ensure that the optimum dose reaches the lungs. Some solutions which are developed for providing feedback on the correct breathing rate to the user are known in the art. In the document numbered US2016256641A1, this is provided by a reed on the airway, and in the document numbered GB2514632A, an inhaler that generates sound in case of appropriate breathing by using a narrowing in the airway or an electronic sound generator has been disclosed.

For determining proper use, measurements of air flow and drug release are also required.

In the document numbered GB2542910A, a system in which a puncture of a drug capsule and air flow were detected by measurements of the corresponding sounds has been disclosed. It has also been stated that the sound measurement is initiated when the inhaler attachment is close to a wearable device on the user from a certain distance.

In document WO2016116629A1, it was disclosed to create an air flow profile by sound measurement (measurement of peak frequencies and frequency band).

In WO2016111633A1, a controller for an inhaler was disclosed. It was stated that if an acoustic signal/acoustic signature related to drug release is received, a record regarding drug release is created. It was also stated that the oscillations made by a sensor a minimum time after determining the opening of the cover and a minimum time before it is closed can be monitored to create the record relating to drug release.

In the document numbered US2017100550A1, an inhaler attachment that controls the usage with sound or vibration measurements has been disclosed. It was provided to measure and eliminate noise even in a noisy environment by means of the two sensors located on the airway. It was also stated that drug-induced sounds can also be measured with this inhaler, and problems such as early drug release, short breathing can be detected by distinguishing drug release from respiration.

In the document numbered WO2009155581A1, a method that provides feedback to the user by examining the sound signals generated by an inhaler was disclosed. It was stated that the feedback can be presented to the user in real time by means of a graph such as time-dependent pressure. It was also explained with this document that the areas corresponding to and not corresponding to suitable usage can be marked on this graph.

In the document numbered GB2484687, capacitance measurements are made when the user touches the inhaler with his/her lips. It is detected that the inhaler will be started to be used with lip contact, and the spray is provided to be pressed automatically even though the user is unable to press the spray.

### SUMMARY

The aim of this invention is related to developing an attachment allowing monitoring inhaler usage of patients. Another aim of the present invention is to develop an inhaler attachment with low energy consumption. With the effective power management system developed by means of this invention, the attachment of the invention can be used for a long period of time without the need for battery replacement.

### BRIEF DESCRIPTION OF THE DRAWINGS

The figures and related descriptions that have been used in order to better explain the power management system of the attachment for monitoring inhaler usage developed with this invention have been described below.

FIG. 1: A schematic perspective view of an exemplary attachment to be used in an inhaler.

FIG. 2: Perspective view of the attachment to be used in an inhaler with inhaler and spray drug.

FIG. 3: A perspective view of the attachment to be used in an inhaler, showing the position of the capacitive measuring ring on the inhaler and with the spray drug in place.

FIG. 4: A perspective view of the attachment to be used in an inhaler, showing the position of the capacitive measuring ring on the inhaler and showing the contact of the user while the spray drug is in place.

FIG. 5: A perspective view of the attachment to be used in an inhaler, showing the position of the capacitive measuring ring on the inhaler in a different location and with the spray drug in place.

FIG. 6: A perspective view of the attachment to be used in an inhaler, showing the position of the capacitive measuring ring on the inhaler in a different location and showing the contact of the user while the spray drug is in place.

FIG. 7: A perspective view of the attachment to be used in an inhaler, showing the position of two capacitive measuring rings on the inhaler and with the spray drug in place.

FIG. 8: A perspective view of the attachment to be used in an inhaler, showing the position of two capacitive measuring rings on the inhaler and the contact of the user with metal spray canister.

FIG. 9: A perspective view of the attachment to be used in an inhaler, showing the position of two capacitive measuring rings on the inhaler and the user pressing the metal spray canister.

FIG. 10: A perspective view of the attachment to be used in an inhaler, showing the position of two capacitive measuring plates on the inhaler and with the spray drug in place.

FIG. 11: A perspective view of the attachment to be used in an inhaler, showing the position of two capacitive measuring plates on the inhaler and the contact of the user with metal spray canister.

FIG. 12: A perspective view of the attachment to be used in an inhaler, showing the position of two capacitive measuring plates on the inhaler and the user pressing the metal spray canister.

FIG. 13: A perspective view of the attachment to be used in an inhaler, showing the position of the capacitive measuring coils on the inhaler and with the spray drug in place.

FIG. 14: A perspective view of the attachment to be used in an inhaler, showing the position of capacitive measuring coils on the inhaler and the contact of the user with metal spray canister.

FIG. 15: A perspective view of the attachment to be used in an inhaler, showing the position of capacitive measuring coils on the inhaler and the user pressing the metal spray canister.

FIG. 16: A side view on the cross-section of an inhaler, showing the positions of the capacitive measuring plates and the metal spray canister of the attachment with the distance between them.

FIG. 17: A side view on the cross-section of the attachment, showing the positions of the capacitive measuring plates and the metal spray canister of the attachment, with the distance between them as the user pushes the canister inside.

FIG. 18: A side view on the cross-section of an inhaler, showing the positions of the capacitive measuring plates and the metal spray canister of the attachment with the distance between them.

FIG. 19: A side view on the cross-section of the attachment, showing the positions of the capacitive measuring plates and the metal spray canister of the attachment, with the distance between them as the user pushes the canister inside.

FIG. 20: A schematic view showing capacitive measurement plates in horizontal lines printed on the printed circuit board.

FIG. 21: A schematic view showing the zigzag capacitive measurement plates printed on the printed circuit board.

FIG. 22: A schematic perspective view of the printed circuit board placed around the inhaler, containing an exemplary capacitive measurement plate included in the attachment to be used in an inhaler.

FIG. 23: A schematic perspective view of the printed circuit board placed around the inhaler, containing an exemplary capacitive measurement plate included in the attachment to be used in an inhaler.

FIG. 24: The schematic perspective view of the attachment to be used in an inhaler, placed on the inhaler.

FIG. 25: The schematic perspective view of an attachment placed on the inhaler, made of flexible material to be used in an inhaler.

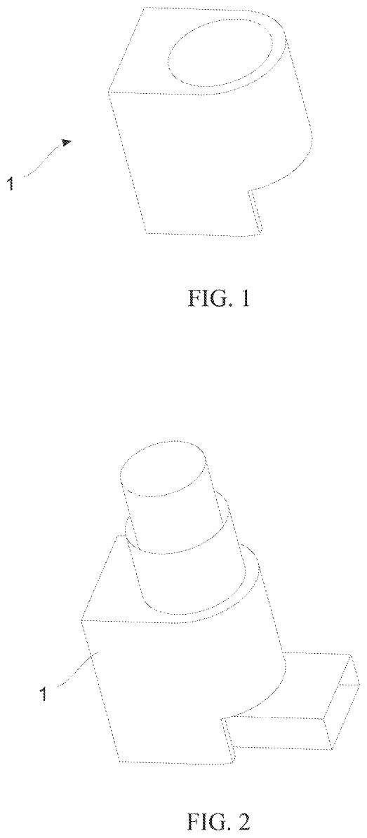

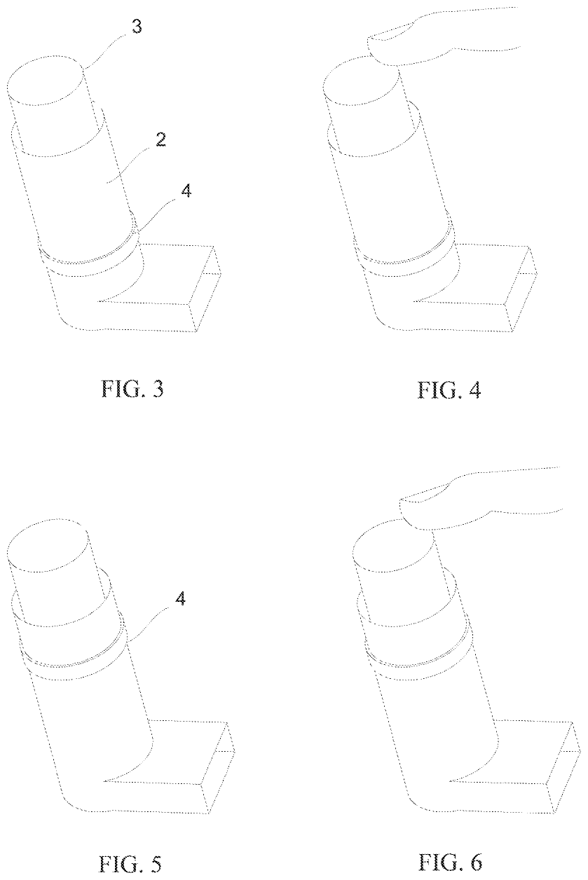

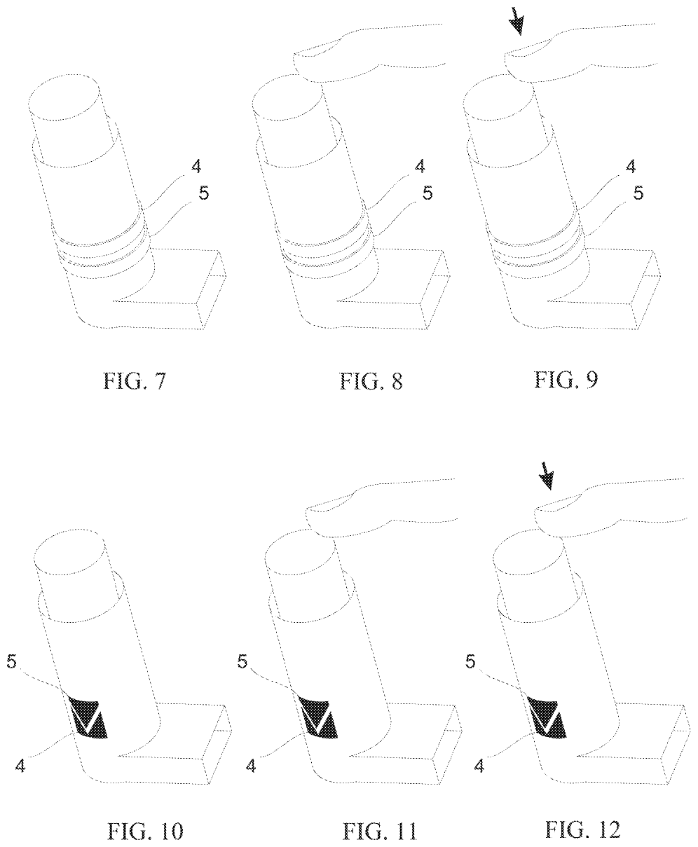

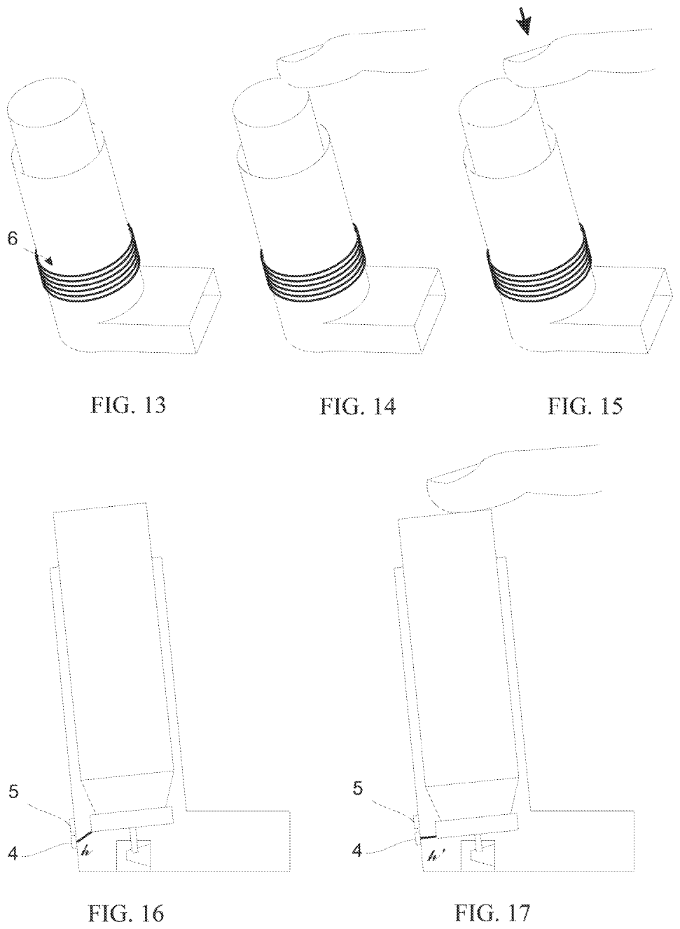

### DEFINITIONS OF THE PARTS OF THE INVENTION

The parts and sections of the inhaler usage monitoring attachment developed with this invention are numbered in the figures and the reference of each number is given below.
1. Attachment
2. Actuator (plastic actuator)
3. Metal spray canister
4. Capacitance sensor
5. Secondary capacitance sensor
6. Capacitance sensor array

### DETAILED DESCRIPTION OF THE EMBODIMENTS

The inventive attachment (1) has been developed to operate with an MDI (Metered Dose Inhaler) inhaler. The MDI inhaler has 3 main components. These are as follows;
A metal spray canister (3); the pressurized drug is stored therein in a solution.
An actuator (2), which ensures that the drug is received from the metal spray canister (3) in aerosol form by being controlled manually.
A dosing valve, which ensures that the same amount of drug, is sprayed in each use.

The attachment (1) moves together with the inhaler by sitting, adhering, grasping, holding onto the plastic actuator (2) of the inhaler, or in other words by being attached onto the actuator. The attachment (1) can be produced either in a form having female-male conformity with actuator (2) or such as to grasp the actuator (2) made from a flexible material or adhere onto it. The attachment (1) has components such as its own power supply and electronic control circuit.

The primary task of the attachment (1) is to record every usage of the inhaler by the user, to store this recording data in the memory thereon and/or to transmit this data to a mobile device in a wired or wireless way. The data on how often the inhaler is used by its user is important in many ways. These can be listed follows;
monitoring of the inhaler and therefore the amount of dose used and the amount that remains in the metal spray canister (3),
monitoring the daily, weekly, monthly and yearly dose of the user,
monitoring usage times and carrying out usage reminders.

In addition to this, whether the user uses the inhaler correctly or not is an important criterion. Because the user should just start to breathe (hand-breath coordination) before 1 dose of drug is sprayed from metal spray canister (3). It is known in the literature that if the drug is sprayed while exhaling or at the end of the breathing period, the effect of the drug received will be less than the optimum effect (sub-optimal drug use).

The attachment (1) can monitor the inhaler usage of the user and/or can also monitor the breathing state and breathing rate. Inhaling and drug spraying activities create vibration and noise on the inhaler. Besides, while the person using the inhaler draws air in, a low pressure environment is created with the air flow formed around the metal spray canister (3).

While these vibrations and sounds can be detected with a microphone, piezo crystal or vibration sensor, the airflow itself can be detected with pressure sensors. Following the detection of the user starting to breath, the time until the drug is sprayed and the monitoring of the time the user stops breathing are important for determining if the drug use is correct or not.

It has been noted above that the user's breathing process can be detected by using any one of the microphone, piezo crystal, vibration sensor or pressure sensors. In addition, since only the action of pressing the inhaler into the metal spray canister (3) can be assumed as use, the detection of the moments of use can also be monitored. For these components to start receiving data only when the user starts using the drug rather than being constantly operated, is a requirement for the battery-powered attachment (1) to be able to save power and thus it can be used for a longer period of time without the need for battery replacement.

Inhalers, in particular certain types of MDI type spray products, are products designed for the use of patients in emergency situations (Rescue Inhaler). For this reason, they are ready to deliver drugs in the most ergonomic structure for the user to access drugs very quickly. In case of emergency or in normal use, it is unacceptable for users to actuate the attachment (1) before accessing the drug and receive the drug after the sensors are ready. For this reason, the sensors that will collect data should be activated before use and the following should be recorded;
the moment of initial breathing of the user,
the moment when the user sprays 1 dose of drug by pressing the metal spray canister (3),
the moment the user stops breathing.

In order to achieve these, the attachment (1) should detect the time when the inhaler will be started to be used and should record the drug use events with its sensors by actuating itself. Components that naturally should come into contact with the hand of the user, when the user grasps the inhaler and puts it into his mouth have been provided. These components are the actuator (2) of the inhaler and the metal spray canister (3). The metal spray canister (3) is produced from aluminum or a similar metal that is resistant to high pressure. This conductive metal spray canister (3) can be detected when it comes close to a capacitance sensor. The conductivity of the metal spray canister (3) can be measured by means of a capacitance sensor (4) which is brought close but is not contacted. Actuators (2) are generally made of plastic, which is an electrically non-conductive material. Thus, if the capacitance sensor (4) is located on the actuator (2) of the inhaler, it is possible to measure the capacitance without contacting the metal spray canister (3).

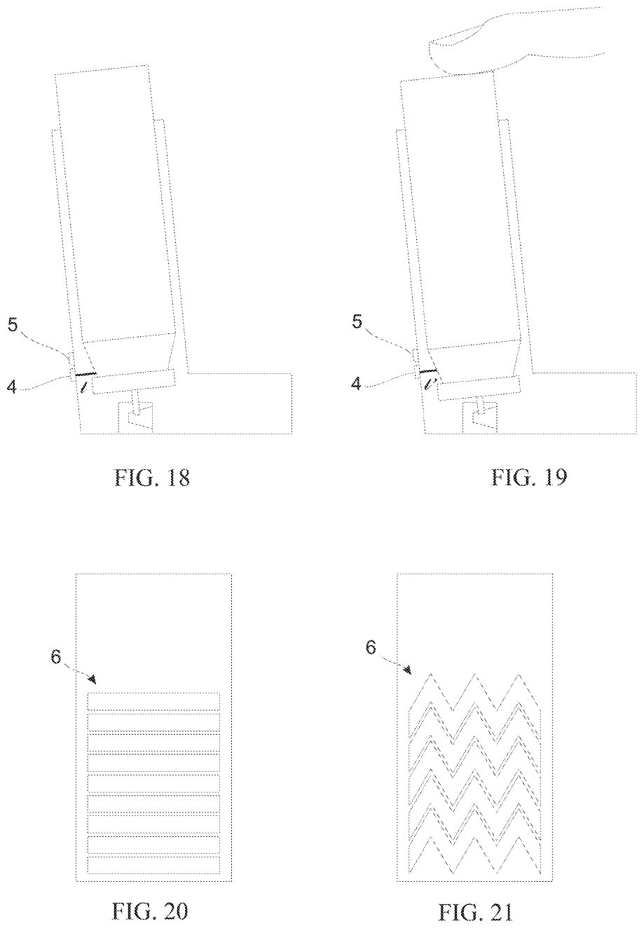

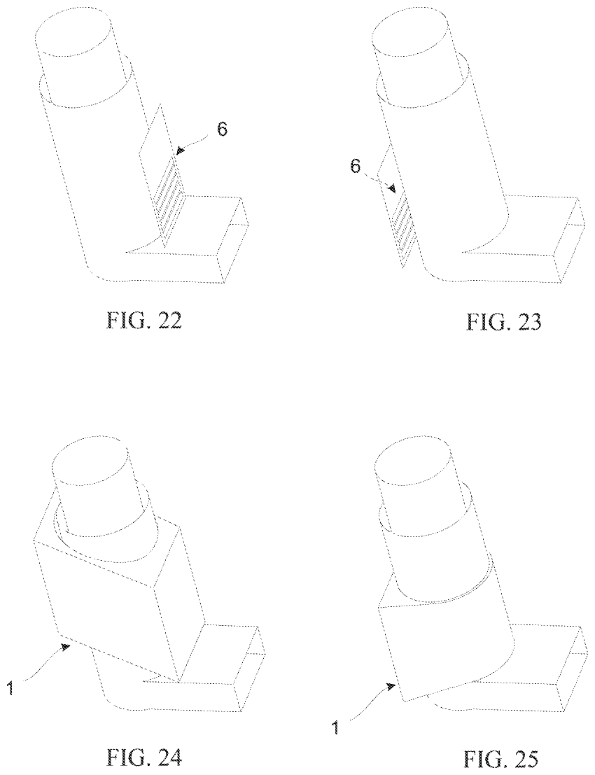

The capacitance value of the metal spray canister (3) varies depending on the amount/shape of the metal contained in this canister and the distance between the capacitance sensor (4) (such as h, l, h′, l′ in FIGS. 16-19). Another factor changing this value is that the user contacts his fingers, which are conductive, with the metal spray canister (3). During this contact, the metal spray canister (3) is grounded by a much larger and conductive object than itself. Moreover, this creates an effect which can be noticed by the capacitance sensor (4) even when the user wearing an insulating glove contacts the metal spray canister (3).

If the capacitance sensor (4) is used alone (self capacitance), the change of the electric charge ratio on this sensor according to the electrical potential of the surrounding system will be monitored. If the capacitance sensor (4) is used together with a secondary capacitance sensor (5), each sensor can perform self-capacitance measurement as well as measure with mutual capacitance method. It is a necessity for the user's hand to contact the metal spray canister (3) for using the inhaler. Because the user ensures that the drug dose is released in a pressurized manner by pressing the metal spray canister (3) into the actuator (2). By this usage, each contact of the user with the metal spray canister (3) can be detected by the capacitance sensor (4). As a result of this detection, it can be possible to actuate the other electronic components and to record inhaler usage data.

It is possible for different conductive objects as well as the user's hand to change the capacitance value measured by the capacitance sensor (4) by contacting the metal spray canister (3). In such cases, the capacitance created by the user's hand can be calibrated according to the amount of change and the duration of the change remaining constant can be checked in order to prevent erroneous initiations.

Although the metal spray canister (3) is cylindrical, it has a changing neck/end structure, i.e., an outer form, near the outlet hole (shown in FIGS. 16-19). If a capacitance sensor (4) is placed in alignment corresponding to the outer form changing towards to tip of the metal spray canister (3), it will be possible to detect capacitance changes from the change of the perceived form when the metal spray canister (3) is pressed against the actuator (2). If the metal spray canister (3) is pressed regardless of its form, the capacitance change originating from the position change can be detected by the capacitance sensor (4) because it will approach or move away from the capacitance sensor (4) by entering into the actuator (2).

An example where the invention is applied was shown in FIGS. 16-19. In these figures, when the metal spray canister (3) moves due to the outer form, its distance to the capacitance sensor (4) (h>>h′ or l>>l′) will change, so the capacitance value will also change.

The metal spray canister (3) does not come to the same position in MDI inhaler actuators (2) marketed by different manufacturers. Therefore, even if it can be measured with a single capacitance sensor (4), in the preferred embodiment of the invention, it is possible to use a secondary capacitance sensor (5) or a capacitance sensor array (6) in the same area.

In particular, if 2 or more capacitance sensors (4) are used, it is inevitable that the movements of the metal spray canister (3) will create a constantly repeating capacitance change profile. This repetitive change profile can be detected algorithmically as well as by machine learning and artificial intelligence techniques. Capacitance sensors (4) will detect not only the user's contact with the metal spray canister (3), but also the spraying of the drug from the metal spray canister (3).

It was stated above that another factor that should be monitored during the use of the inhaler is the respiration of the user. Still, in the preferred embodiment of the invention, the time the user starts and stops breathing is detected by means of changes in air pressure, but not by sound and vibration. For this purpose, the use of the form of the metal spray canister (3) and the correct inhaler usage technique is beneficial.

During the spraying of the drug from the metal spray canister (3), in order for the optimum dose to reach the lungs, the air flow inhaled by the user should be within the breathing rate range determined by the drug manufacturer as much as possible. For this purpose, air enters into the inhaler around the metal spray canister (3) and is directed towards the outlet that is held by the lips of the user.

As the air enters the inhaler rapidly from the periphery of the metal spray canister (3), it causes the pressure to drop. This pressure drop will be recorded as the moment of breathing initiation by being detected by a pressure sensor located on the attachment (1).

If the user stops breathing, the pressure sensor will detect a higher pressure value due to the air slowing down or even stagnating. The moment when this high pressure value is detected will be recorded as the time when breathing ends.

Capacitance sensors (4) are already used within touchless buttons. In the practice of the present invention, the detection of the moment of use and/or the starting of the other electronic components in the attachment (2) will be provided by means of the capacitance sensor (4). The exemplary operation algorithms of the attachment (1) have been specified below. These are as follows;

The operation algorithm of the attachment (1) comprising a single capacitance sensor (4);
After the capacitance sensor (4) measures the capacitance value of the metal spray canister (3) for the period of “t1”, if there is no increase in the capacitance value due to the hand contact of the user with the metal spray canister (3), it will turn off for the period of “t2”.
After the capacitance sensor (4) measures the capacitance value of the metal spray canister (3) for a period of “t1”, if the metal spray canister (3) is in contact with the user's hand, it will continue to measure the capacitance during the “t3” period without turning off itself.
During capacitance measurement, if the user pushes the metal spray canister (3) into the actuator (2), since the value of h>>h′ or l>>l′ will change, the capacitance sensor (4) will detect that the metal spray canister (3) is spraying 1 dose of drug.
When the user releases the metal spray canister (3), the metal spray canister (3) will come to its first position and this situation can also be detected by the capacitance sensor (4).

The operation algorithm of the attachment (1) comprising a capacitance sensor (4) and a secondary capacitance sensor (5);
After the capacitance sensor (4) and/or secondary capacitance sensor (5) measures the capacitance value of the metal spray canister (3) for the period of “t1”, if there is no increase in the capacitance value due to the hand contact of the user with the metal spray canister (3), it will turned off for the period of “t2”.
After the capacitance sensor (4) and/or the secondary capacitance sensor (5) measures the capacitance value of the metal spray canister (3) for the period of “t1”, if the metal spray canister (3) is in contact with the hand of the user, both the capacitance sensor (4) and the secondary capacitance sensor (5) will continue to perform the capacitance measurement for a period of “t3” without turning itself off.
During capacitance measurement, if the user pushes the metal spray canister (3) into the actuator (2), since the value of h>>h′ or l>>l′ will change, the capacitance sensor (4) and/or secondary capacitance sensor (5) will detect that the metal spray canister (3) is spraying 1 dose of drug.
When the user releases the metal spray canister (3), the metal spray canister (3) will come to its first position and this situation can also be detected by the capacitance sensor (4) and/or the secondary capacitance sensor (5).

The operation algorithm of the attachment (1) comprising a capacitance sensor array (6);
After the capacitance sensor array (6) measures the capacitance value of the metal spray canister (3) for the period of “t1”, if there is no increase in the capacitance value due to the hand contact of the user with the metal spray canister (3), it will turn off for the period of “t2”.
After the capacitance sensor array (6) measures the capacitance value of the metal spray canister (3) for a period of “t1”, if the metal spray canister (3) is in contact with the user's hand, it will continue to perform the capacitance measurement with capacitance sensor array (6) during the “t3” period without turning off itself.
During capacitance measurement, if the user pushes the metal spray canister (3) into the actuator (2), since the value of h>>h′ or l>>l′ will change, the capacitance sensor array (6) will detect that the metal spray canister (3) is spraying 1 dose of drug.
When the user releases the metal spray canister (3), the metal spray canister (3) will come to its first position and this situation can also be detected by the capacitance sensor array (6).

If the attachment (1) includes a sensor (for example, pressure sensor, vibration sensor) that will measure the air flow as well as capacitance sensors (4), the exemplary algorithms for turning this sensor on and off are given below. These are as follows;

The operation algorithm of the attachment (1) comprising a single capacitance sensor (4) and air flow measurement sensor;
After the capacitance sensor (4) measures the capacitance value of the metal spray canister (3) for the period of “t1”, if there is no increase in the capacitance value due to the hand contact of the user with the metal spray canister (3), it will turn off for 1 second.
After the capacitance sensor (4) measures the capacitance value of the metal spray canister (3) for a period of “t2”, if the metal spray canister (3) is in contact with the user's hand, it will continue to measure capacitance for 2 minutes without turning off itself and at the same time, it will open the turned off air flow measurement sensor for the period of “t3”.
During capacitance measurement, if the user pushes the metal spray canister (3) into the actuator (2), since the value of h>>h′ or l>>l′ will change, the capacitance sensor (4) will detect that the metal spray canister (3) is spraying 1 dose of drug.
The air flow sensor will record the user's breathing by monitoring vibrations and/or air pressure while it is open.
The user's breathing and whether the spraying times are suitable for correct use will be recorded by being detected.
When the user releases the metal spray canister (3), the metal spray canister (3) will come to its first position and this situation can also be detected by the capacitance sensor (4).

The working algorithm of the attachment (1) comprising a capacitance sensor (4), the secondary capacitance sensor (5) and air flow measurement sensor;
After the capacitance sensor (4) and/or secondary capacitance sensor measures the capacitance value of the metal spray canister (3) for the period of “t1”, if there is no increase in the capacitance value due to the hand contact of the user with the metal spray canister (3), it will turned off for the period of “t2”.
After the capacitance sensor (4) and/or the secondary capacitance sensor (5) measure the capacitance value of the metal spray canister (3) for the period of “t1”, if the metal spray canister (3) is in contact with the hand of the user, both capacitance sensor (4) and the secondary capacitance sensor (5) will continue to perform the capacitance measurement and at the same time, it will open the turned off air measurement sensor for 2 minutes.
During capacitance measurement, if the user pushes the metal spray canister (3) into the actuator (2), since the value of h>>h′ or l>>l′ will change, the capacitance sensor (4) and/or secondary capacitance sensor (5) will detect that the metal spray canister (3) is spraying 1 dose of drug.
The air flow sensor will record the user's breathing by monitoring vibrations and/or air pressure while it is open.
The user's breathing and whether the spraying times are suitable for correct use will be recorded by being detected.
When the user releases the metal spray canister (3), the metal spray canister (3) will come to its first position and this situation can also be detected by the capacitance sensor (4) and/or the secondary capacitance sensor (5).

The working algorithm of the attachment (1) comprising a capacitance sensor array (6) and air flow measurement sensor;
After the capacitance sensor array (6) measures the capacitance value of the metal spray canister (3) for the period of “t1”, if there is no increase in the capacitance value due to the hand contact of the user with the metal spray canister (3), it will turn off for the period of “t2”.
After the capacitance sensor array (6) measures the capacitance value of the metal spray canister (3) for a period of “t1”, if the metal spray canister (3) is in contact with the user's hand, it will continue to perform the capacitance measurement with capacitance sensor array (6) during the “t3” period without turning off itself and at the same time, it will open the turned off air flow sensor for 2 minutes.
During capacitance measurement, if the user pushes the metal spray canister (3) into the actuator (2), since the value of h>>h′ or l>>l′ will change, the capacitance sensor array (6) will detect that the metal spray canister (3) is spraying 1 dose of drug.
The air flow sensor will record the user's breathing by monitoring vibrations and/or air pressure while it is open.
The user's breathing and whether the spraying times are suitable for correct use will be recorded by being detected.
When the user releases the metal spray canister (3), the metal spray canister (3) will come to its first position and this situation can also be detected by the capacitance sensor array (6).

As described in detail above, the inventive attachment (1) will detect both the moment when the user is holding the inhaler in his/her hand for use and the moment of drug spraying by measuring the capacitance. Furthermore, when the user grasps the inhaler for use, the pressure sensor or microphone or vibration sensor that has remained closed for reasons such as energy saving and electronic circuit elements that control them will be activated.

Any or all of the capacitance sensor (4) or secondary capacitance sensor (5) or the capacitance sensor arrays (6) which perform capacitance measurement can be present in the attachment (1) according to the invention such that it will perform the self capacitance measurement. If any or all of these are used, it is sufficient to have only one capacitance sensor (4) open continuously and to monitor the capacitance value of the metal spray canister (3) to determine whether the user is in contact with the metal spray canister (3).

Similar to the description of the invention above, in cases where a capacitance sensor (4) and a secondary capacitance sensor (5) are used together, it is possible for these capacitance sensors to measure the capacitance value by forming a mutual capacitor (mutual capacitance). In such applications of the invention, it is sufficient for the pair of capacitance sensors (4 and 5) that continuously form a capacitor, that is, the mutual capacitance sensor, to be open and to monitor, the capacitance value of the metal spray canister (3) to understand whether the user is in contact with the metal spray canister (3).

Metal spray canister (3) holds medicine in gas or liquid form. In order to monitor the amount of gas/liquid mixture that decreases after each use, the sounds made by the metal spay canister (3) during shaking, the amount of vibration, and the shaking character can be monitored by microphone or vibration censor.

What is claimed is:

1. An attachment, comprising a self-capacitance sensor measuring a capacitance change of a metal spray canister when a user contacts the metal spray canister to detect a moment of the metal spray canister starts to be used, wherein an inhaler for monitoring a use of patients using the inhaler comprises:
the metal spray canister, wherein in the metal spray canister, a drug is stored in a solution in a pressurized way;
an actuator for ensuring the drug is received from the metal spray canister in an aerosol form by being controlled manually.

2. The attachment according to claim 1, wherein the attachment detects the capacitance change occurring due to a change of a position of the metal spray canister while moving relative to the actuator.

3. The attachment according to claim 1, wherein the attachment detects the capacitance change occurring since the aerosol form detected by the attachment has changed while the metal spray canister moves relative to the actuator.

4. The attachment according to claim 2, comprising a self-capacitance sensor array, wherein the self-capacitance sensor array detects the capacitance change instead of detecting the capacitance change with only one self-capacitance sensor, during a movement of the metal spray canister relative to the actuator.

5. The attachment according to claim 3, comprising a self-capacitance sensor array, wherein the self-capacitance sensor array is used instead of the self-capacitance sensor for detecting a change of the aerosol form while the metal spray canister is moving relative to actuator.

6. The attachment according to claim 1, comprising a pressure sensor, wherein the pressure sensor monitors a pressure drop around the metal spray canister to monitor a breathing of the user.

7. The attachment according to claim 1, comprising a microphone, a piezo crystal or a vibration sensor monitoring a sound or vibrations to monitor a breathing of the user.

8. The attachment according to claim 1, wherein the self-capacitance sensor determines a contact of the user to the metal spray canister and activates a pressure sensor or a microphone or a vibration sensor and electronic circuit elements controlling the pressure sensor or the microphone or the vibration sensor.

9. The attachment according to claim 4, wherein the self-capacitance sensor opens the self-capacitance sensor array following a contact by the user.

10. An attachment, comprising a mutual capacitance sensor, wherein a capacitance value of the mutual capacitance sensor has changed prior to a contact in a case a user contacts to a metal spray canister to detect a moment of the metal spray canister starts to be used, wherein an inhaler for monitoring a use of patients using the inhaler comprises:
the metal spray canister, wherein in the metal spray canister the drug is stored in a solution in a pressurized manner;
an actuator for ensuring the drug is received from the metal spray canister in an aerosol form by being controlled manually.

11. The attachment according to claim 10, wherein the attachment is configured for detecting a capacitance value change occurring due to a change of a position of the metal spray canister while moving relative to the actuator.

12. The attachment according to claim 10, wherein the attachment is configured for detecting a capacitance value change occurring since the aerosol form detected by the attachment has changed while the metal spray canister moves relative to the actuator.

13. The attachment according to claim 11, wherein the change of the position of the metal spray canister is monitored by a mutual capacitance sensor array instead of only one mutual capacitance sensor when the metal spray canister moves relative to the actuator.

14. The attachment according to claim 12, comprising a mutual capacitance sensor array, wherein the mutual capacitance sensor array is used instead of only one mutual capacitance sensor for detecting a change of the aerosol form while metal spray canister is moving relative to actuator.

15. The attachment according to claim 10, comprising a pressure sensor, wherein the pressure sensor tracks a pressure drop around the metal spray canister to monitor a breathing of the user.

16. The attachment according to claim 10, comprising a microphone, a piezo crystal or a vibration sensor monitoring a sound or vibrations to monitor a breathing of the user.

17. The attachment according to claim 10, wherein the mutual capacitance sensor measures the capacitance change by detecting the contact of the user to the metal spray canister, and activating a pressure sensor or a microphone or a vibration sensor and electronic circuit elements controlling the the pressure sensor or the microphone or the vibration sensor.

18. The attachment according to claim 13, wherein the mutual capacitance sensor measures a mutual capacitance change, and the mutual capacitance sensor opens the mutual capacitance sensor array following the contact of the user.
